(19) United States
(12) Patent Application Publication
(10) Pub. No.: US 2026/0294680 A1
(43) Pub. Date: Oct. 1, 2026

(54) SURFACE MODIFIED OPHTHALMIC DEVICES

(71) Applicant: Bausch + Lomb Ireland Limited, Dublin (IE)

(72) Inventors: Alok Kumar Awasthi, Pittsford, NY (US); Sankarsan Biswas, Rochester, NY (US); Feng-Yang Shih, Lafayette, CO (US); Jade Russell, Perry, NY (US); James Anthony DiBella, JR., Penfield, NY (US)

(21) Appl. No.: 19/564,584

(22) Filed: Mar. 12, 2026

Related U.S. Application Data
(60) Provisional application No. 63/780,716, filed on Mar. 31, 2025.

(51) Int. Cl. A61F 9/00; A61K 31/4725; A61K 31/728; C08F 251/00

(52) U.S. Cl. CPC A61F 9/0017; A61K 31/4725; A61K 31/728; C08F 251/00

(57) ABSTRACT

A surface modified ophthalmic device includes an ophthalmic device having a plurality of surface functional groups and a surface coating, the surface coating having a lifitegrast or a salt thereof-containing monomer or a lifitegrast or a salt thereof-containing polymer attached to a surface of the ophthalmic device through the plurality of surface functional groups.

### PRIORITY CLAIM

The present application claims priority to U.S. Provisional Patent Application Ser. No. 63/780,716, entitled “Surface Modified Ophthalmic Devices,” filed Mar. 31, 2025, the content of which is incorporated by reference herein in its entirety.

### BACKGROUND

Ophthalmic devices such as contact lenses made from, for example, silicone-containing materials, have been investigated for a number of years. Such materials can generally be subdivided into two major classes, namely hydrogels and non-hydrogels. Hydrogels can absorb and retain water in an equilibrium state, whereas non-hydrogels do not absorb appreciable amounts of water. Regardless of their water content, both hydrogel and non-hydrogel silicone medical devices tend to have relatively hydrophobic, non-wettable surfaces that have a high affinity for lipids. This problem is of particular concern with contact lenses.

Those skilled in the art have long recognized the need for modifying the surface of the silicone ophthalmic devices such as silicone contact lenses so that they are compatible with the eye. For example, by increasing the hydrophilicity of a contact lens surface, the wettability of the contact lens can be improved. This, in turn, is associated with improved wear comfort of the contact lenses. Additionally, the surface of the lens can affect the lens's susceptibility to deposition, particularly the deposition of proteins and lipids resulting from tear fluid during lens wear. Accumulated deposition can cause eye discomfort or even inflammation. In the case of extended wear lenses (i.e., lenses used without daily removal of the lens before sleep), the surface is especially important, since extended wear lenses must be designed for high standards of comfort and biocompatibility over an extended period of time.

### SUMMARY

In accordance with an illustrative embodiment, a surface modified ophthalmic device comprises an ophthalmic device having a plurality of surface functional groups and a surface coating, the surface coating comprising a lifitegrast or a salt thereof-containing monomer or a lifitegrast or a salt thereof-containing polymer attached to a surface of the ophthalmic device through the plurality of surface functional groups.

In accordance with another illustrative embodiment, a method for making a surface modified ophthalmic device comprises:
providing an ophthalmic device having a plurality of surface functional groups, and
attaching a lifitegrast or a salt thereof-containing monomer or a lifitegrast or a salt thereof-containing polymer to a surface of the ophthalmic device through the plurality of surface functional groups.

In accordance with yet another illustrative embodiment, a method for delivering lifitegrast or a salt thereof to an eye of a subject comprises:
placing a surface modified ophthalmic device in an eye of a subject, wherein the surface modified ophthalmic device comprises an ophthalmic device having a plurality of surface functional groups and a surface coating, the surface coating comprising a lifitegrast or a salt thereof-containing monomer or a lifitegrast or a salt thereof-containing polymer attached to a surface of the ophthalmic device through the plurality of surface functional groups, and
delivering the lifitegrast or salt thereof to the eye of the subject by contacting the surface modified ophthalmic device with one of light having a wavelength of about 300 nanometers (nm) to about 700 nm or with an esterases or enzymes on the eye, in a tear film or on a flora of the eye.

In accordance with yet another illustrative embodiment, a packaging system for the storage of a surface modified ophthalmic device comprises a sealed container containing an unused ophthalmic device having a plurality of surface functional groups immersed in an aqueous packaging solution comprising a lifitegrast or a salt thereof-containing monomer or a lifitegrast or a salt thereof-containing polymer, wherein the aqueous packaging solution has an osmolality of at least about 150 mOsm/kg, a pH of about 6 to about 9 and is sterilized.

In accordance with still yet another illustrative embodiment, a method of preparing a packaging system comprising a storable, sterile surface modified ophthalmic device comprises:
(a) providing an unused ophthalmic device having a plurality of surface functional groups,
(b) immersing the unused ophthalmic device having the plurality of surface functional groups in an aqueous packaging solution comprising a lifitegrast or a salt thereof-containing monomer or a lifitegrast or a salt thereof-containing polymer, wherein the aqueous packaging solution has an osmolality of at least about 150 mOsm/kg and a pH in the range of about 6 to about 9,
(c) packaging the aqueous packaging solution and the unused ophthalmic device in a manner preventing contamination of the unused ophthalmic device by microorganisms, and
(d) sterilizing the packaged solution and unused ophthalmic device.

### DETAILED DESCRIPTION

Various illustrative embodiments described herein include surface modified ophthalmic devices comprising an ophthalmic device having a plurality of surface functional groups and a surface coating, the surface coating comprising a lifitegrast or a salt thereof-containing monomer or a lifitegrast or a salt thereof-containing polymer attached to a surface of the ophthalmic device through the plurality of surface functional groups.

### Definitions

A “prepolymer” refers to a starting polymer which can be cured (e.g., crosslinked and/or polymerized) actinically or thermally or chemically to obtain a crosslinked and/or polymerized polymer having a molecular weight higher than the molecular weight of the starting polymer.

A “crosslinkable prepolymer” refers to a starting polymer which can be crosslinked to obtain a crosslinked polymer having a molecular weight higher than the molecular weight of the starting polymer.

Representative examples of ethylenically unsaturated reactive groups for use herein include, by way of example, a (meth)acrylate-containing reactive end group, a (meth)acrylamide-containing reactive end group, an acrylate-containing reactive end group, an acrylamide-containing reactive end group, an allyl-containing reactive end group, a vinyl-containing reactive end group, a vinylcarbonate-containing reactive end group, a vinylcarbamate-containing reactive end group, a styrene-containing reactive end group, an itaconate-containing reactive end group, a vinyloxy-containing reactive end group, a fumarate-containing reactive end group, a maleimide-containing reactive end group, a vinylsulfonyl reactive end group and the like.

In a non-limiting illustrative embodiment, a (meth)acrylate-containing reactive end group can be represented by the structure:

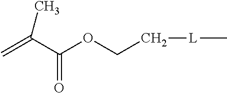

wherein L is a linking group or bond. Suitable linking groups include, for example, a heteroatom such as 0, any divalent hydrocarbon radical or moiety such as independently a straight or branched, substituted or unsubstituted C₁-C₆ alkyl group, a substituted or unsubstituted C₃-C₁₂ cycloalkyl group, a substituted or unsubstituted C₄-C₁₂ cycloalkylalkyl group, a substituted or unsubstituted C₃-C₁₂ cycloalkenyl group, a substituted or unsubstituted C₆-C₁₂ aryl group, a substituted or unsubstituted C₇-C₁₂ arylalkyl group and substituted and unsubstituted ether-containing groups.

As used herein, the term “(meth)” denotes an optional methyl substituent. Thus, terms such as “(meth)acrylate” denote either methacrylate or acrylate, and “(meth)acrylamide” denotes either methacrylamide or acrylamide.

While compositions and processes are described in terms of “comprising” various components or steps, the compositions and processes can also “consist essentially of” or “consist of” the various components or steps, unless stated otherwise.

The terms “a,” “an,” and “the” are intended to include plural alternatives, e.g., at least one. The terms “including,” “with,” and “having,” as used herein, are defined as comprising (i.e., open language), unless specified otherwise.

Various numerical ranges are disclosed herein. When Applicant discloses or claims a range of any type, Applicant's intent is to disclose or claim individually each possible number that such a range could reasonably encompass, including end points of the range as well as any sub-ranges and combinations of sub-ranges encompassed therein, unless otherwise specified. For example, all numerical end points of ranges disclosed herein are approximate, unless excluded by proviso.

Values or ranges may be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In another aspect, use of the term “about” means±20% of the stated value, ±15% of the stated value, ±10% of the stated value, ±5% of the stated value, ±3% of the stated value, or ±1% of the stated value.

Applicant reserves the right to proviso out or exclude any individual members of any such group of values or ranges, including any sub-ranges or combinations of sub-ranges within the group, that can be claimed according to a range or in any similar manner, if for any reason Applicant chooses to claim less than the full measure of the disclosure, for example, to account for a reference that Applicant may be unaware of at the time of the filing of the application. Further, Applicant reserves the right to proviso out or exclude any members of a claimed group.

Ophthalmic drug delivery faces significant obstacles due to anatomical and physiological barriers, such as tear drainage and limited epithelial transport, which restrict drug bioavailability to around 5% or less. These barriers impede the penetration and absorption of therapeutic agents, making effective treatment challenging. An optimal delivery system must address these issues by enhancing bioavailability and enabling controlled drug release at the target site.

As used herein, the term “ophthalmic device” refers to devices that reside in or on the eye. These ophthalmic devices can provide optical correction, wound care, drug delivery, diagnostic functionality or cosmetic enhancement or effect or a combination of these properties. Representative examples of such devices include, but are not limited to, soft contact lenses, e.g., a soft, hydrogel lens; soft, non-hydrogel lens and the like, hard contact lenses, e.g., a hard, gas permeable lens material and the like, intraocular lenses, overlay lenses, ocular inserts, optical inserts and the like. As is understood by one skilled in the art, a lens is considered to be “soft” if it can be folded back upon itself without breaking. A contact lens can be in a dry state or a wet state. A “dry state” refers to a soft contact lens in a state prior to hydration or the state of a hard lens under storage or use conditions. A “wet state” refers to a soft contact lens in a hydrated state.

In general, ophthalmic devices such as contact lenses are intended for direct contact with body tissue or body fluid, e.g., direct contact in the eye. It is highly desirable that an ophthalmic device such as a contact lens be as comfortable as possible for wearers. Manufacturers of contact lenses are continually working to improve the comfort of the lenses. Nevertheless, many people who wear contact lenses still experience dryness or eye irritation throughout the day and particularly towards the end of the day. An insufficiently wetted lens at any point in time will cause significant discomfort to the lens wearer. Although wetting drops can be used as needed to alleviate such discomfort, it would certainly be desirable if such discomfort did not arise in the first place.

Lifitegrast or salts thereof are well-established medications for the treatment of dry eye disease. Ophthalmic lifitegrast is specifically used to alleviate the signs and symptoms associated with this condition. Classified as a lymphocyte function-associated antigen-1 (LFA-1) antagonist, lifitegrast or salt thereof-containing monomer or polymer functions by reducing inflammation in the eye tissues.

Conventional methods like eye drops and ointments have been used to deliver lifitegrast. However, these delivery methods are inefficient, requiring frequent applications and often resulting in poor patient compliance and incorrect dosing. While advanced formulations such as gels, viscous solutions, and colloidal systems have been developed, they have yet to achieve satisfactory outcomes.

Accordingly, these problems are overcome by the illustrative embodiments disclosed herein by providing an improved surface modified ophthalmic devices such as a surface modified contact lens having a surface coating, the surface coating comprising a lifitegrast or a salt thereof-containing monomer or a lifitegrast or a salt thereof-containing polymer attached to a surface of the ophthalmic device through a plurality of surface functional groups such that the surface modified ophthalmic device would be more lubricious and comfortable to wear in actual use thereby allowing for extended wear of the surface modified ophthalmic device without irritation or other adverse effects to the cornea. When the surface modified ophthalmic device is worn by a user, the lifitegrast or salt thereof can be delivered to the eye of the user in a continuous and sustained release manner. By providing significantly greater drug bioavailability to the cornea compared to the traditional methods discussed above, the surface modified ophthalmic devices described herein containing the the surface coating comprising a lifitegrast or a salt thereof-containing monomer or a lifitegrast or a salt thereof-containing polymer attached to a surface of the ophthalmic device through a plurality of surface functional groups can be utilized as a controlled drug delivery platform. In addition, when the lifitegrast or salt thereof is attached to a hydrophilic polymer such as hyaluronic acid, the ophthalmic device can provide improved lubricity and/or wettability of the lens and comfort to the eye. The surface modified ophthalmic device will therefore be even more comfortable to wear in actual use and allow for further extended wear of the lens without adverse effects to the cornea.

The non-limiting illustrative embodiments disclosed herein overcome the foregoing drawbacks by providing a surface modified ophthalmic device comprising an ophthalmic device having a plurality of surface functional groups and a surface coating, the surface coating comprising a lifitegrast or a salt thereof-containing monomer or a lifitegrast or a salt thereof-containing polymer attached to a surface of the ophthalmic device through the plurality of surface functional groups. For example, the surface coating attached to a surface of the ophthalmic device comprises a lifitegrast or a salt thereof-containing monomer comprising a releasable linking group or a lifitegrast or a salt thereof-containing polymer comprising a releasable linking group, the releasable linking group linking the lifitegrast or salt thereof-containing monomer or the lifitegrast or salt thereof-containing polymer to a complementary reactive functionality of the ophthalmic device.

The surface modified ophthalmic devices according to the non-limiting illustrative embodiments described herein advantageously deliver lifitegrast or a salt thereof to the eye of a user in a continuous and sustained release manner. The lifitegrast or salt thereof can be delivered to an eye of a subject by contacting the surface modified ophthalmic device with one of light having a wavelength of about 300 nanometers (nm) to about 700 nm or with an esterases or enzymes on the eye, in a tear film or on a flora of the eye thereby releasing the lifitegrast or salt thereof in a sustained release manner.

### Ophthalmic Device

The type of ophthalmic device to be coated with the surface coating disclosed herein is not critical and any ophthalmic device is contemplated. The ophthalmic device can be any material known in the art capable of forming an ophthalmic device. In some embodiments, an ophthalmic device includes devices which are formed from material not hydrophilic per se. Such devices are formed from materials known in the art and include, by way of example, polysiloxanes, perfluoropolyethers, fluorinated poly(meth)acrylates or equivalent fluorinated polymers derived, e.g., from other polymerizable carboxylic acids, polyalkyl(meth)acrylates or equivalent alkylester polymers derived from other polymerizable carboxylic acids, or fluorinated polyolefins, such as fluorinated ethylene propylene polymers, or tetrafluoroethylene, preferably in combination with a dioxol, e.g., perfluoro-2,2-dimethyl-1,3-dioxol. Representative examples of suitable bulk materials include, but are not limited to, lotrafilcon A, neofocon, pasifocon, telefocon, silafocon, fluorsilfocon, paflufocon, silafocon, elastofilcon, fluorofocon or Teflon® AF materials, such as Teflon® AF 1600 or Teflon® AF 2400 which are copolymers of about 63 to about 73 mol % of perfluoro-2,2-dimethyl-1,3-dioxol and about 37 to about 27 mol % of tetrafluoroethylene, or of about 80 to about 90 mol % of perfluoro-2,2-dimethyl-1,3-dioxol and about 20 to about 10 mol % of tetrafluoroethylene.

In some embodiments, an ophthalmic device includes a device which is formed from material hydrophilic per se, since reactive groups, e.g., carboxy, carbamoyl, sulfate, sulfonate, phosphate, amine, ammonium or hydroxy groups, are inherently present in the material and therefore also at the surface of an ophthalmic device manufactured therefrom. Suitable hydrophilic monomers include, for example, unsaturated carboxylic acids, acrylamides, vinyl lactams, hydroxyl-containing-(meth)acrylates, hydrophilic vinyl carbonates, hydrophilic vinyl carbamates, hydrophilic oxazolones, and poly(alkene glycols) functionalized with polymerizable groups and the like and mixtures thereof. Representative examples of unsaturated carboxylic acids include, but are not limited to, methacrylic acid, acrylic acid and the like and mixtures thereof. Representative examples of acrylamides include, but are not limited to, alkylamides such as N,N-dimethylacrylamide, N,N-dimethylmethacrylamide and the like and mixtures thereof. Representative examples of cyclic lactams include, but are not limited to, N-vinyl-2-pyrrolidone, N-vinyl caprolactam, N-vinyl-2-piperidone and the like and mixtures thereof. Representative examples of hydroxyl-containing (meth)acrylates include, but are not limited to, 2-hydroxyethyl methacrylate (HEMA), glycerol methacrylate and the like and mixtures thereof.

Additional hydrophilic monomers include, for example, the hydrophilic vinyl carbonate or vinyl carbamate monomers disclosed in U.S. Pat. No. 5,070,215, and the hydrophilic oxazolone monomers disclosed in U.S. Pat. No. 4,910,277. Other suitable silicone hydrogel-forming hydrophilic comonomers will be apparent to one skilled in the art. Mixtures of the foregoing hydrophilic comonomers can also be used in the ophthalmic device-forming mixtures herein.

In accordance with one or more additional non-limiting illustrative embodiments, as may be combined with one or more of the preceding paragraphs, the one or more hydrophilic comonomers can be present in a monomeric mixture in a major amount, e.g., an amount greater than 50 wt. %, based on the total weight of the monomeric mixture. In some embodiments, the one or more hydrophilic comonomers can be present in a monomeric mixture in an amount ranging from about 20 wt. % to about 60 wt. %, based on the total weight of the monomeric mixture. In another illustrative embodiment, the one or more hydrophilic comonomers can be present in the monomeric mixture in an amount ranging from about 25 wt. % to about 45 wt. %, based on the total weight of the monomeric mixture.

In another embodiment, an ophthalmic device includes a device which is formed from materials which are amphiphilic segmented copolymers containing at least one hydrophobic segment and at least one hydrophilic segment which are linked through a bond or a bridge member.

It is particularly useful to employ biocompatible materials herein including both soft and rigid materials commonly used for ophthalmic lenses, including contact lenses. In general, non-hydrogel materials are hydrophobic polymeric materials that do not contain water in their equilibrium state. Typical non-hydrogel materials comprise silicone acrylics, such as those formed from a bulky silicone monomer (e.g., tris(trimethylsiloxy)silylpropyl methacrylate, commonly known as “TRIS” monomer), methacrylate end-capped poly(dimethylsiloxane)prepolymer, or silicones having fluoroalkyl side groups (polysiloxanes are also commonly known as silicone polymers).

In another embodiment, an ophthalmic device includes a device which is a hydrogel. Hydrogels in general are a well-known class of materials that comprise hydrated, crosslinked polymeric systems containing water in an equilibrium state. Accordingly, hydrogels are copolymers prepared from hydrophilic monomers such as those described above. In the case of silicone hydrogels, the hydrogel copolymers are generally prepared by polymerizing a mixture containing at least one silicone-containing monomer and at least one hydrophilic monomer. Either the silicone-containing monomer or the hydrophilic monomer can function as a crosslinking agent (a crosslinker being defined as a monomer having multiple polymerizable functionalities) or a separate crosslinker may be employed. Silicone hydrogels typically have a water content between about 10 wt. % to about 80 wt. %.

The monomer mixtures may also include a second monomer including a copolymerizable group and a reactive functional group. The copolymerizable group is preferably an ethylenically unsaturated group, such that this monomer copolymerizes with the hydrophilic device-forming monomer and any other device-forming monomers in the initial device-forming monomer mixture. Additionally, the second monomer can include a reactive functional group that reacts with a complementary reactive group of the copolymer such as the reaction product of one or more polymerizable polyhydric alcohols and one or more polymerizable fluorine-containing monomers. In other words, after the device is formed by copolymerizing the monomeric mixture, the reactive functional groups provided by the second monomers remain to react with a complementary reactive moiety of the copolymer.

In an embodiment, reactive groups of the second monomers include epoxide groups. Accordingly, second monomers are those that include both an ethylenically unsaturated group (that permits the monomer to copolymerize with the hydrophilic device-forming monomer) and the epoxide group (that does not react with the hydrophilic monomer but remains to react with a copolymer, e.g., the reaction product of one or more polymerizable polyhydric alcohols and one or more polymerizable fluorine-containing monomers). Suitable second monomers include, for example, glycidyl methacrylate, glycidyl acrylate, glycidyl vinylcarbonate, glycidyl vinylcarbamate, and 4-vinyl-1-cyclohexene-1,2-epoxide.

As mentioned, one class of ophthalmic device materials are silicone hydrogels. In this case, the initial monomeric mixture further comprises a silicone-containing monomer. Applicable silicone-containing monomeric materials for use in the formation of silicone hydrogels are well known in the art and numerous examples are provided in U.S. Pat. Nos. 4,136,250; 4,153,641; 4,740,533; 5,034,461; 5,070,215; 5,260,000; 5,310,779; and 5,358,995. Specific examples of suitable materials for use herein include those disclosed in U.S. Pat. Nos. 5,310,779; 5,387,662; 5,449,729; 5,512,205; 5,610,252; 5,616,757; 5,708,094; 5,710,302; 5,714,557 and 5,908,906, the contents of which are incorporated by reference herein.

In some embodiments, a class of representative silicone-containing monomers include, for example, bulky siloxane monomers having an ethylenically unsaturated reactive group. The term “bulky” refers to groups on the siloxane monomer that are sterically and/or electronically encumbering, i.e., sterically hindering. In a non-limiting illustrative embodiment, suitable bulky siloxane monomers include, for example, a bulky polysiloxanylalkyl (meth)acrylic monomer, a bulky polysiloxanylalkyl carbamate monomer and mixtures thereof. Representative examples of applicable silicone-containing monomers include bulky polysiloxanylalkyl(meth)acrylic monomers. In one embodiment, a representative example of a bulky siloxane monomer is represented by a structure of Formula Ia:

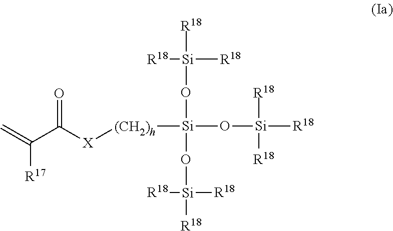

wherein X denotes —O— or —NR¹⁹—, where each R¹⁹ is hydrogen or a C₁-C₄ alkyl group; R¹⁷ independently denotes hydrogen or methyl; each R¹⁸ independently denotes a lower alkyl radical such as a C₁-C₆ group, a phenyl radical or a group represented by the following structure:

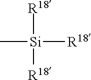

wherein each R^{18′} independently denotes a lower alkyl radical such as a C₁-C₆ group or a phenyl radical; and h is 1 to 10; or is represented by a structure of Formula Ib:

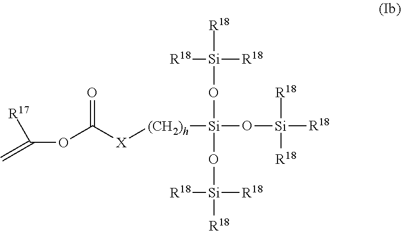

wherein X denotes —NR¹⁹ wherein R¹⁹ denotes hydrogen or a C₁-C₄ alkyl; R¹⁷ denotes hydrogen or methyl; each R¹⁸ independently denotes a lower alkyl radical such as a C₁-C₆ group, a phenyl radical or a group represented by the following structure:

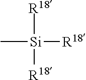

wherein each R^{18′} independently denotes a lower alkyl radical such as a C₁-C₆ group or a phenyl radical; and h is 1 to 10.

Representative examples of bulky siloxane monomers include 3-methacryloyloxypropyltris(trimethylsiloxy)silane ortris(trimethylsiloxy)silylpropyl methacrylate, sometimes referred to as TRIS and tris(trimethylsiloxy)silylpropyl vinyl carbamate, sometimes referred to as TRIS-VC, pentamethyldisiloxanyl methylmethacrylate, phenyltetramethyl-disiloxanylethyl acetate, and methyldi(trimethylsiloxy)methacryloxymethyl silane, (3-methacryloxy-2-hydroxy propoxy)propyl bis(trimethyl siloxy)methyl silane, sometimes referred to as Sigma and the like and mixtures thereof. In one embodiment, the bulky siloxane monomer is a tris(trialkylsiloxy)silylalkyl methacrylate-containing monomer such as a tris(trimethylsiloxy)silylpropyl methacrylate-containing monomer.

Another class of representative silicone-containing monomers includes, for example, silicone-containing vinyl carbonate or vinyl carbamate monomers such as, for example, 1,3-bis[4-vinyloxycarbonyloxy)but-1-yl]tetramethyl-disiloxane; 3-(trimethylsilyl)propyl vinyl carbonate; 3-(vinyloxycarbonylthio)propyl-[tris(trimethylsiloxy)silane]; 3-[tris(trimethylsiloxy)silyl]propyl vinyl carbamate; 3-[tris(trimethylsiloxy)silyl]propyl allyl carbamate; 3-[tris(trimethylsiloxy)silyl]propyl vinyl carbonate; t-butyldimethylsiloxyethyl vinyl carbonate; trimethylsilylethyl vinyl carbonate; trimethylsilylmethyl vinyl carbonate and the like and mixtures thereof.

Another class of silicone-containing monomers includes polyurethane-polysiloxane macromonomers (also sometimes referred to as prepolymers), which may have hard-soft-hard blocks like traditional urethane elastomers. They may be end-capped with a hydrophilic monomer such as HEMA. Examples of such silicone urethanes are disclosed in a variety or publications, including Lai, Yu-Chin, “The Role of Bulky Polysiloxanylalkyl Methacryates in Polyurethane-Polysiloxane Hydrogels,” Journal of Applied Polymer Science, Vol. 60, 1193-1199 (1996). PCT Published Application No. WO 96/31792 discloses examples of such monomers, which disclosure is hereby incorporated by reference in its entirety. Further examples of silicone urethane monomers are represented by Formula II and III:

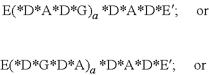

wherein:
   D independently denotes an alkyl diradical, an alkyl cycloalkyl diradical, a cycloalkyl diradical, an aryl diradical or an alkylaryl diradical having 6 to about 30 carbon atoms;
   G independently denotes an alkyl diradical, a cycloalkyl diradical, an alkyl cycloalkyl diradical, an aryl diradical or an alkylaryl diradical having 1 to about 40 carbon atoms and which may contain ether, thio or amine linkages in the main chain;
   * denotes a urethane or ureido linkage;
   a is at least 1;
   A independently denotes a divalent polymeric radical of Formula IV:

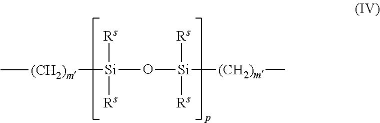

wherein each R^{s} independently denotes an alkyl or fluoro-substituted alkyl group having 1 to about 10 carbon atoms which may contain ether linkages between the carbon atoms; m′ is at least 1; and p is a number that provides a moiety weight of about 400 to about 10,000;
   each of E and E′ independently denotes a polymerizable unsaturated organic radical represented by Formula V:

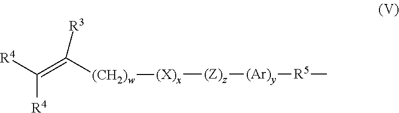

wherein: R³ is hydrogen or methyl;
R⁴ is hydrogen, an alkyl radical having 1 to 6 carbon atoms, or a —CO—Y—R⁶ radical wherein
Y is —O—, —S— or —N—H—;
R⁵ is a divalent alkylene radical having 1 to about 10 carbon atoms;
R⁶ is an alkyl radical having 1 to about 12 carbon atoms;
X denotes —CO— or —OCO—;
Z denotes —O— or —NH—;
Ar denotes an aromatic radical having about 6 to about 30 carbon atoms;
w is 0 to 6; x is 0 or 1; y is 0 or 1; and z is 0 or 1.

In an embodiment, a silicone-containing urethane monomer is represented by Formula VI:

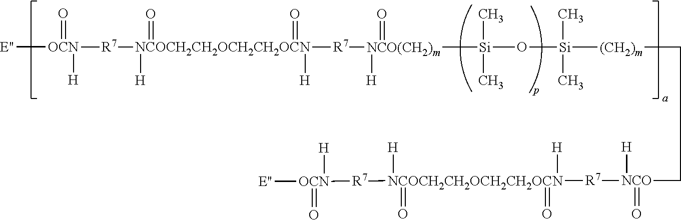

wherein m is at least 1 and is preferably 3 or 4, a is at least 1 and preferably is 1, p is a number which provides a moiety weight of about 400 to about 10,000 and is preferably at least about 30, R⁷ is a diradical of a diisocyanate after removal of the isocyanate group, such as the diradical of isophorone diisocyanate, and each E″ is a group represented by:

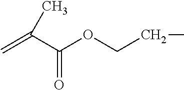

In another embodiment, a silicone hydrogel material comprises (in bulk, that is, in the monomer mixture that is copolymerized) about 5 wt. % to about 50 wt. %, or from about 10 wt. % to about 25 wt. % of one or more silicone macromonomers, about 5 wt. % to about 75 wt. %, or about 30 wt. % to about 60 wt. % of one or more polysiloxanylalkyl (meth)acrylic monomers, and about 10 wt. % to about 50 wt. %, or about 20 wt. % to about 40 wt. % of a hydrophilic monomer. In general, the silicone macromonomer is a poly(organosiloxane) capped with an unsaturated group at two or more ends of the molecule. In addition to the end groups in the above structural formulas, U.S. Pat. No. 4,153,641 discloses additional unsaturated groups, including acryloxy or methacryloxy. Fumarate-containing materials such as those disclosed in U.S. Pat. Nos. 5,310,779; 5,449,729 and 5,512,205 are also useful substrates in accordance with the invention. The silane macromonomer may be a silicon-containing vinyl carbonate or vinyl carbamate or a polyurethane-polysiloxane having one or more hard-soft-hard blocks and end-capped with a hydrophilic monomer.

In another embodiment, a class of silicon-containing monomers includes monomers of Formula VII:

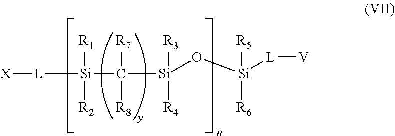

wherein X is the residue of a ring opening agent; L is the same or different and is a linker group or a bond; V is an ethylenically unsaturated polymerizable group; R₁, R₂, R₃, R₄, R₅, R₆ are independently H, alkyl, halo alkyl, cyclo alkyl, heterocyclo alkyl, alkenyl, halo alkenyl, or aromatic; R₇ and R₈ are independently H or alkyl wherein at least one of R₇ or R₈ is hydrogen; y is 2 to 7 and n is 1 to 100.

Ring opening agents are well known in the literature. Non-limiting examples of anionic ring opening agents include alkyl lithiums, alkoxides, trialkylsiloxylithium wherein the alkyl group may or may not contain halo atoms.

Linker groups can be any divalent radical or moiety and include substituted or unsubstituted alkyl, alkyl ether, alkenyls, alkenyl ethers, halo alkyls, substituted or unsubstituted siloxanes, and monomers capable of propagating ring opening.

Ethylenically unsaturated polymerizable groups are well known to those skilled in the art. Non-limiting examples of ethylenically unsaturated polymerizable groups would include acrylates, methacrylates, vinyl carbonates, O-vinyl carbamates, N-vinyl carbamates, acrylamides and methacrylamides.

In another embodiment, a class of silicone-containing monomers includes monomers of Formula VIII:

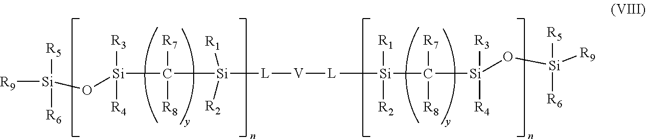

wherein L is the same or different and is a linker group or a bond; V is the same or different and is an ethylenically unsaturated polymerizable group; R₁, R₂, R₃, R₄, R₅, R₆ and R₉ are independently H, alkyl, halo alkyl, cyclo alkyl, heterocyclo alkyl, alkenyl, halo alkenyl, or aromatic; R₇ and R₈ are independently H or alkyl wherein at least one of R₇ or R₈ is hydrogen; y is 2-7 and n is 1-100.

In another embodiment, a class of silicone-containing monomers includes monomers of Formulas IX and X:

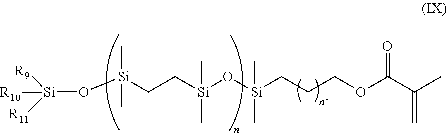

wherein R₉, R₁₀ and R₁₁ are independently H, alkyl, haloalkyl or other substituted alkyl groups; n is as defined above and n¹ is 0-10; and,

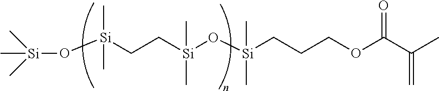

wherein n is 1 to 100, or n is 2 to 80, or n is 3 to 20, or n is 5 to 15.

In another embodiment, a class of silicon-containing monomers includes monomers of Formulas XI-XV:

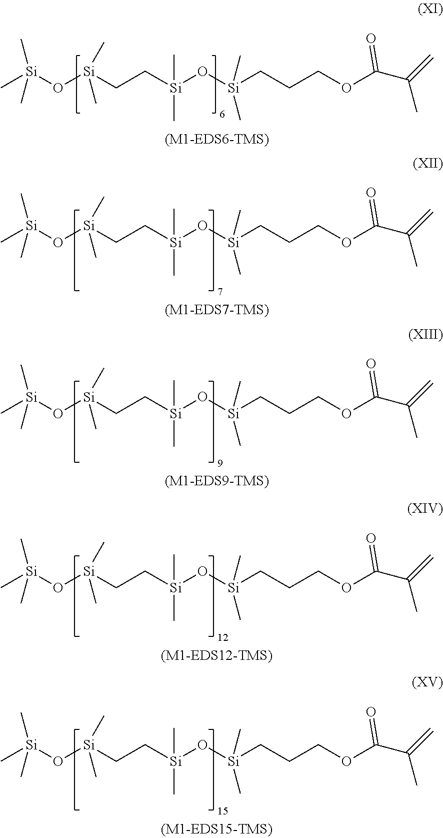

In another embodiment, a class of silicone-containing monomers includes monomers of Formulas XVI-XVIII:

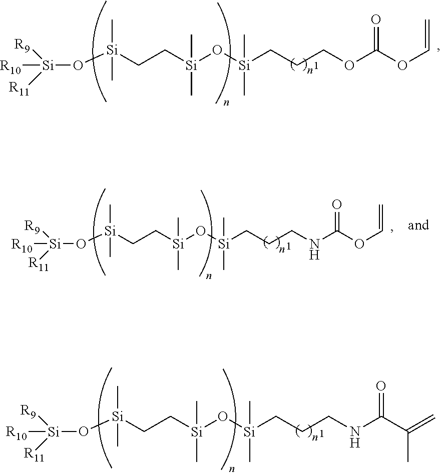

wherein R₉, R₁₀ and R₁₁ are independently H, alkyl, haloalkyl or other substituted alkyl groups and n and n¹ are as defined above.

In another embodiment, a class of silicone-containing monomers includes monomers of Formulas XIX-XXI:

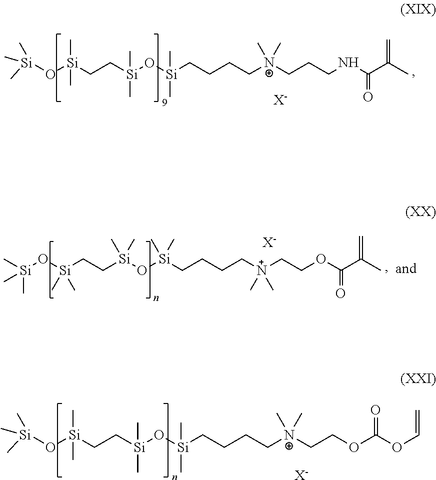

wherein n is as defined above and X⁻ is a counterion to provide an overall neutral charge.

Counterions capable of providing an overall neutral charge are well known to those of ordinary skill in the art and would include, for example, halide ions.

In another embodiment, a class of silicone-containing monomers includes monomers of Formula XXII:

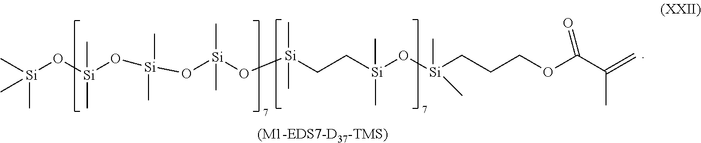

Another class of representative silicone-containing monomers includes fluorinated monomers. Such monomers have been used in the formation of fluorosilicone hydrogels to reduce the accumulation of deposits on contact lenses made therefrom, as disclosed in, for example, U.S. Pat. Nos. 4,954,587; 5,010,141 and 5,079,319. Also, the use of silicone-containing monomers having certain fluorinated side groups, i.e., —(CF₂)—H, have been found to improve compatibility between the hydrophilic and silicone-containing monomeric units. See, e.g., U.S. Pat. Nos. 5,321,108 and 5,387,662.

Another class of representative silicone-containing monomers includes, for example, a monofunctional silicone monomer represented by a structure of Formula XXIII:

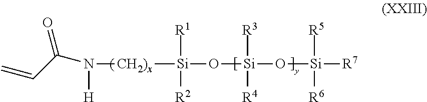

wherein R¹, R², R³ and R⁴ are independently hydrogen, an alkyl group, a halo alkyl group, a cycloalkyl group, a heterocycloalkyl group, an alkenyl group, a haloalkenyl group, an aryl group and a heteroaryl group; R⁵, R⁶ and R⁷ are independently a straight or branched alkyl group; x is from 1 to 6; and y is from 3 to 15.

In some embodiments, R¹, R², R³ and R⁴ of the monofunctional silicone monomer represented by a structure of Formula XXIII are independently hydrogen, a C₁ to C₁₂ alkyl group, a C₁ to C₁₂ halo alkyl group, a C₃ to C₁₂ cycloalkyl group, a C₃ to C₁₂ heterocycloalkyl group, a C₂ to C₁₂ alkenyl group, a C₂ to C₁₂ haloalkenyl group, a C₆ to C₁₂ aromatic group and a C₆ to C₁₂ heteroaromatic group; R⁵, R⁶ and R⁷ are independently a straight or branched C₁ to C₁₂ alkyl group; x is from 1 to 6; and y is from 3 to 8.

In some embodiments, R¹, R², R³ and R⁴ of the monofunctional silicone monomer represented by a structure of Formula XXIII are independently hydrogen, a C₁ to C₆ alkyl group; R⁵, R⁶ and R⁷ are independently a straight or branched C₁ to C₆ alkyl group; x is from 1 to 6; and y is from 3 to 8.

In some embodiments, R¹, R², R³ and R⁴ are independently a C₁ to C₃ alkyl group; R⁵ and R⁶ are independently a C₁ to C₃ alkyl group; R⁷ is a straight or branched C₃ to C₆ alkyl group; x is from 2 to 4; and y is from 3 to 15.

Representative examples of alkyl groups for use herein include, by way of example, a straight or branched alkyl chain radical containing carbon and hydrogen atoms of from 1 to about 30 carbon atoms or from 1 to about 12 carbon atoms or from 1 to about 6 carbon atoms with or without unsaturation, to the rest of the molecule, e.g., methyl, ethyl, n-propyl, 1-methylethyl (isopropyl), n-butyl, n-pentyl, methylene, ethylene, etc., and the like, optionally containing one or more heteroatoms, e.g., O and N, and the like, or one or more halogen atoms, e.g., fluorine, chlorine, bromine, and iodine, to form a halo alkyl group.

Representative examples of cycloalkyl groups for use herein include, by way of example, a substituted or unsubstituted, non-aromatic mono or multicyclic ring system of about 3 to about 30 carbon atoms or from 3 to about 12 carbon atoms or from 3 to about 6 carbon atoms such as, for example, cyclopropyl, cyclobutyl, cyclopentyl, cyclohexyl, perhydronapththyl, adamantyl and norbornyl groups, bridged cyclic groups or sprirobicyclic groups, e.g., spiro-(4, 4)-non-2-yl and the like, optionally containing one or more heteroatoms, e.g., O and N, and the like to form a heterocycloalkyl group.

Representative examples of cycloalkylalkyl groups for use herein include, by way of example, a substituted or unsubstituted, cyclic ring-containing radical containing from about 4 to about 30 carbon atoms or from 3 to about 6 carbon atoms directly attached to the alkyl group which is then attached to the main structure of the monomer at any carbon from the alkyl group that results in the creation of a stable structure such as, for example, cyclopropylmethyl, cyclobutylethyl, cyclopentylethyl and the like, wherein the cyclic ring can optionally contain one or more heteroatoms, e.g., O and N, and the like to form a heterocycloalkylalkyl group.

Representative examples of cycloalkenyl groups for use herein include, by way of example, a substituted or unsubstituted cyclic ring-containing radical containing from about 3 to about 30 carbon atoms or from 3 to about 6 carbon atoms with at least one carbon-carbon double bond such as, for example, cyclopropenyl, cyclobutenyl, cyclopentenyl and the like, wherein the cyclic ring can optionally contain one or more heteroatoms, e.g., O and N, and the like to form a heterocycloalkenyl group.

Representative examples of aryl groups for use herein include, by way of example, a substituted or unsubstituted, monoaromatic or polyaromatic radical containing from about 6 to about 30 carbon atoms or from about 6 to about 12 carbon atoms such as, for example, phenyl, naphthyl, tetrahydronapthyl, indenyl, biphenyl and the like, optionally containing one or more heteroatoms, e.g., O and N, and the like to form a heteroaryl group.

In an illustrative embodiment, the monofunctional silicone monomer represented by the structure of Formula XXIII is either commercially available from such sources as ShinEtsu or can be made by methods within the purview of one skilled in the art. For example, in an illustrative embodiment, the monofunctional silicone monomer represented by the structure of Formula XXIII can be prepared according to the following reaction Scheme I.

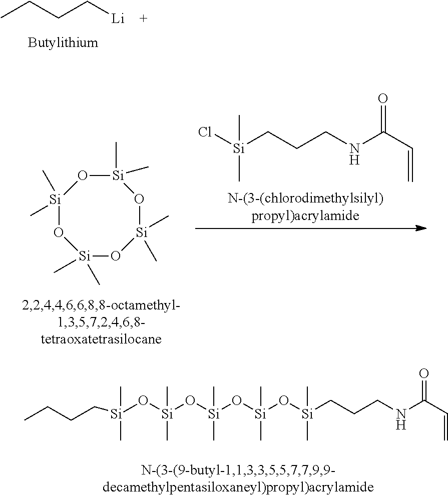

In some embodiments, the one or more monofunctional silicone monomers represented by a structure of Formula XXIII as disclosed herein can be present in the ophthalmic device-forming mixture in an amount ranging from about 5 wt. % to about 40 wt. %, based on the total weight of the ophthalmic device-forming mixture. In some embodiments, the one or more silicone monomers represented by a structure of Formula XXIII as disclosed herein can be present in the ophthalmic device-forming mixture in an amount ranging from about 7 wt. % to about 15 wt. %, based on the total weight of the ophthalmic device-forming mixture.

The above silicone materials are merely exemplary, and other materials for use as ophthalmic devices that have been disclosed in various publications and are being continuously developed for use in contact lenses and other ophthalmic devices can also be used. For example, an ophthalmic device can be formed from at least a cationic monomer such as cationic silicone-containing monomer or cationic fluorinated silicone-containing monomers.

Ophthalmic devices such as contact lenses can be manufactured by employing various conventional techniques, to yield a shaped article having the desired posterior and anterior lens surfaces, e.g., by spincasting and static casting methods. Spincasting methods are disclosed in U.S. Pat. Nos. 3,408,429 and 3,660,545; and static casting methods are disclosed in U.S. Pat. Nos. 4,113,224, 4,197,266 and 5,271,876. Spincasting methods involve charging the monomeric mixtures to be polymerized to a mold, and spinning the mold in a controlled manner while exposing the mixture to a radiation source such as UV light. Static casting methods involve charging the monomeric mixtures between two mold sections, one mold section shaped to form the anterior lens surface and the other mold section shaped to form the posterior lens surface, and curing the ophthalmic device-forming mixture while retained in the mold assembly to form an ophthalmic device, for example, by free radical polymerization of the ophthalmic device-forming mixture. Curing of the monomeric mixture may be followed by a machining operation in order to provide a contact lens having a desired final configuration. As an example, U.S. Pat. No. 4,555,732 discloses a process in which an excess of a monomeric mixture is cured by spincasting in a mold to form a shaped article having an anterior lens surface and a relatively large thickness. The posterior surface of the cured spincast article is subsequently lathe cut to provide a contact lens having the desired thickness and posterior lens surface. Further machining operations may follow the lathe cutting of the lens surface, for example, edge-finishing operations.

### Surface Coating

In non-limiting illustrative embodiments, as may be combined with one or more of the preceding paragraphs, the foregoing ophthalmic devices having a plurality of surface functional groups can be surface modified by attaching a lifitegrast or a salt thereof-containing monomer or a lifitegrast or a salt thereof-containing polymer to a surface of the ophthalmic device through the plurality of surface functional groups to form a surface coating. In some embodiments, the plurality of surface functional groups are selected from the group consisting of a hydroxy group, amino group, carboxy group, carbonyl group, aldehyde group, sulfonic acid group, sulfonyl chloride group, isocyanato group, carboxy anhydride group, lactone group, azlactone group, epoxy group and mixtures thereof.

In general, a lifitegrast-containing monomer can be represented by the following structure:

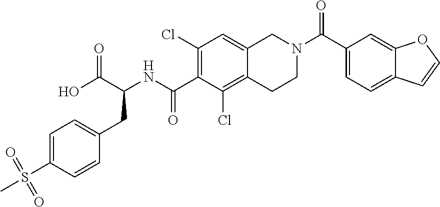 [SMILES: CS(=O)(=O)c1ccc(C[C@H](NC(=O)c2c(Cl)cc3c(c2Cl)CCN(C(=O)c2ccc4ccoc4c2)C3)C(=O)O)cc1]

and a lifitegrast salt-containing monomer can be represented by the following structure:

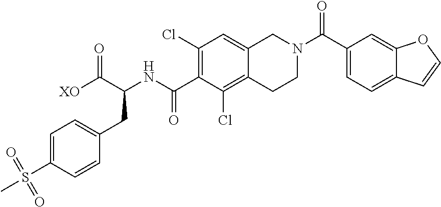

where x is a salt such as a sodium salt.

In some embodiments, the lifitegrast or salt thereof-containing monomer can be attached to the plurality of surface functional groups of the ophthalmic device via a reactive functionality of the lifitegrast or salt thereof-containing monomer. For example, a reactive functionality of the lifitegrast or salt thereof-containing monomer can include, for example, a carboxylic acid group or a carboxylate group. Upon attaching the reactive functionality of the lifitegrast or salt thereof-containing monomer to the plurality of surface functional groups of the ophthalmic device, a releasable linking group is formed that includes at least one bond that can be broken under physiological conditions, e.g., where the releasable linking group is a group linking the lifitegrast or salt thereof-containing monomer to the plurality of surface functional groups of the ophthalmic device via a pH-labile bond, an acid-labile bond, a base-labile bond, an oxidatively labile bond, a metabolically labile bond, a biochemically labile bond or an enzyme-labile bond, or other conditions such as a standard chemical reaction via a chemical-labile bond, e.g., a hydrolysis or substitution reaction, or via a photo-labile bond, e.g., exposure to light such as UV, near UV or visible light, e.g., a photosensitive bond. If the releasable linking groups are naturally cleaved under physiological conditions or cellular physiological conditions, then the monomer is biodegradable. In some embodiments, the releasable linking groups results in a loss of the lifitegrast or salt thereof from the surface of the ophthalmic device thereby releasing the lifitegrast or salt thereof.

In some embodiments, the releasable linking group includes ester groups. In the case of ester bonds, the esters undergo hydrolysis and are also catalytically cleaved by esterases thereby releasing the lifitegrast or salt thereof.

In some embodiments, a lifitegrast or a salt thereof-containing polymer can includes, for example, a lifitegrast or a salt thereof-containing glycosaminoglycan (GAG) polymer comprising a GAG having a polymer backbone and a side chain comprising a lifitegrast or a salt thereof residue grafted onto the polymer backbone. In other words, a reactive functional group in the polymer backbone is grafted onto a reactive functional group of lifitegrast or a salt thereof such as a carboxylic acid group or a carboxylate group. A GAG is one molecule with many alternating subunits. In general, GAGs are represented by the formula A-B-A-B-A-B, where A is uronic acid and B is an amino sugar that may or may not be either O- or N-sulfated, where the A and B units can be heterogeneous with respect to epimeric content or sulfation. Any natural or synthetic polymer containing uronic acid can be used. Other GAGs are sulfated at different sugars. There are many different types of GAGs having commonly understood structures such as, for example, chondroitin sulfate (e.g., chondroitin 4- and 6-sulfates), heparan, heparin sulfate, heparosan, dermatan, dermatan sulfate, hyaluronic acid or a salt thereof, e.g., sodium hyaluronate or potassium hyaluronate, keratan sulfate, and other disaccharides such as sucrose, lactulose, lactose, maltose, trehalose, cellobiose, mannobiose and chitobiose. GAGs can be purchased from Sigma, and many other biochemical suppliers such as HTL Biotechnology (France). In an illustrative embodiment, the GAG is hyaluronic acid. In another illustrative embodiment, the GAG is chondroitin sulfate.

In an illustrative embodiment, a GAG for use herein can have a weight average molecular weight ranging from about 10,000 to about 3,000,000 Daltons (Da) in which the lower limit is from about 10,000, about 20,000, about 30,000, about 40,000, about 50,000, about 60,000, about 70,000, about 80,000, about 90,000, or about 100,000 Da, and the upper limit is about 200,000, about 300,000, about 400,000, about 500,000, about 600,000, about 700,000, about 800,000, about 900,000, about 1,000,000, or about up to 2,800,000 Da, where any of the lower limits can be combined with any of the upper limits. In an illustrative embodiment, a GAG for use herein can have a weight average molecular weight ranging from about 1,000,000 to about 3,000,000 Da.

Hyaluronic acid is a well-known, naturally occurring, water soluble biodegradable polymer composed of two alternatively linked sugars, D-glucuronic acid and N-acetylglucosamine, linked via alternating β-(1,4) and β-(1,3) glycosidic bonds. Hyaluronic acid is a non-sulfated GAG. The polymer is hydrophilic and highly viscous in aqueous solution at relatively low solute concentrations. It often occurs naturally as the sodium salt, sodium hyaluronate. Methods of preparing commercially available hyaluronan and salts thereof are well known. Hyaluronan can be purchased from Seikagaku Company, Clear Solutions Biotech, Inc., Pharmacia Inc., Sigma Inc., HTL Biotechnology, Contipro and Bloomage Biotechnology Corporation, and many other suppliers. Hyaluronic acid has repeating units of the structure represented by the following formula:

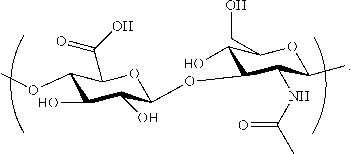

Accordingly, the repeating units in hyaluronic acid can be as follows:

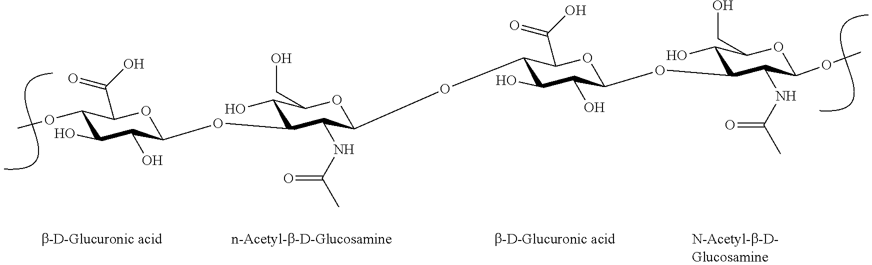

In general, hyaluronic acid or a salt thereof can have from about 2 to about 1,500,000 disaccharide units. In an embodiment, hyaluronic acid or a salt thereof can have a weight average molecular weight ranging from about 10,000 to about 3,000,000 Da in which the lower limit is from about 10,000, about 20,000, about 30,000, about 40,000, about 50,000, about 60,000, about 70,000, about 80,000, about 90,000, or about 100,000 Da, and the upper limit is about 200,000, about 300,000, about 400,000, about 500,000, about 600,000, about 700,000, about 800,000, about 900,000, about 1,000,000, or about up to 2,800,000 Da, where any of the lower limits can be combined with any of the upper limits.

Chondroitin sulfate is a linear sulfated polysaccharide composed of repeating β-D-glucuronic acid (GlcA) and N-acetyl-β-D-galactosamine (GalNAc) units arranged in the sequence by GlcA-β(1,3)-GalNAc-β(1,4) glycosidic bonds. In an embodiment, chondroitin sulfate has one or more repeating units of the structure represented by the following formula:

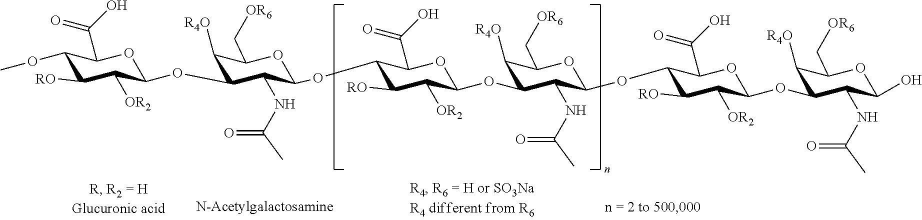

In an illustrative embodiment, chondroitin sulfate has repeating units of the structure represented by the following formula:

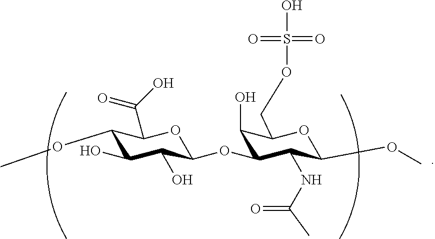

In general, chondroitin sulfate can have from about 2 to about 1,500,000 repeating units. In an embodiment, chondroitin sulfate can have a weight average molecular weight ranging from about 10,000 to about 3,000,000 Da in which the lower limit is from about 5,000, 10,000, about 20,000, about 30,000, about 40,000, about 50,000, about 60,000, about 70,000, about 80,000, about 90,000, or about 100,000 Da, and the upper limit is about 200,000, about 300,000, about 400,000, about 500,000, about 600,000, about 700,000, about 800,000, about 900,000, about 1,000,000, or about 3,000,000 Da where any of the lower limits can be combined with any of the upper limits or any of the upper limits can be combined with any of the upper limits.

In an illustrative embodiment, dermatan sulfate has repeating units of the structure represented by the following formula:

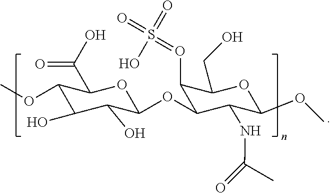

In general, dermatan sulfate can have from about 2 to about 1,500,000 repeating units. In an embodiment, dermatan sulfate can have a weight average molecular weight ranging from about 1,000 to about 2,000,000 Da in which the lower limit is from about 1,000, 5,000, 10,000, about 20,000, about 30,000, about 40,000, about 50,000, about 60,000, about 70,000, about 80,000, about 90,000, or about 100,000 Da, and the upper limit is about 200,000, about 300,000, about 400,000, about 500,000, about 600,000, about 700,000, about 800,000, about 900,000, about 1,000,000, or about 2,000,000 Da where any of the lower limits can be combined with any of the upper limits or any of the upper limits can be combined with any of the upper limits.

In an illustrative embodiment, heparin and heparin sulfate have repeating units of the structure represented by the following formula:

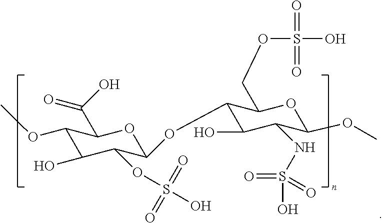

In general, heparin and heparin sulfate can have from about 2 to about 1,500,000 repeating units. In an embodiment, heparin and heparin sulfate can have a weight average molecular weight ranging from about 1,000 to about 3,000,000 Da in which the lower limit is from about 1,000, 5,000, 10,000, about 20,000, about 30,000, about 40,000, about 50,000, about 60,000, about 70,000, about 80,000, about 90,000, or about 100,000 Da, and the upper limit is about 40,000, 100,000, 200,000, about 300,000, about 400,000, about 500,000, about 600,000, about 700,000, about 800,000, about 900,000, about 1,000,000, or about 3,000,000 Da where any of the lower limits can be combined with any of the upper limits or any of the upper limits can be combined with any of the upper limits.

In an illustrative embodiment, keratan sulfate has repeating units of the structure represented by the following formula:

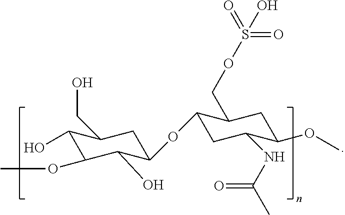

In general, keratan sulfate can have from about 2 to about 1,500,000 repeating units. In an embodiment, keratan sulfate can have a weight average molecular weight ranging from about 10,000 to about 3,000,000 Da in which the lower limit is from about 5,000, 10,000, about 20,000, about 30,000, about 40,000, about 50,000, about 60,000, about 70,000, about 80,000, about 90,000, or about 100,000 Da, and the upper limit is about 100,000, 200,000, about 300,000, about 400,000, about 500,000, about 550,000, 600,000, about 700,000, about 800,000, about 900,000, about 1,000,000, or about 3,000,000 Da where any of the lower limits can be combined with any of the upper limits or any of the upper limits can be combined with any of the upper limits.

The lifitegrast or salt thereof-containing GAG polymers disclosed herein can be obtained by grafting a reactive end group of the lifitegrast or salt thereof onto a complementary reactive functionality in the polymer backbone of the GAG. For example, in one illustrative embodiment, a carboxylic acid group or a carboxylate group of the lifitegrast or salt thereof can be grafted onto a carboxylic acid group in the polymer backbone of the GAG.

In general, the GAG and the lifitegrast or salt thereof can be added sequentially or simultaneously to a reaction mixture. The reaction can be carried out at a suitable temperature and for a time period for the completion of the reaction to maximize the yield of the product lifitegrast or salt thereof-containing residue grafted onto the polymer backbone of the GAG. For example, a suitable temperature and time period includes a temperature ranging from about 10 to about 40° C. and a time period ranging from about 4 hours to about 48 hours. In an illustrative embodiment, a suitable temperature and time period includes a temperature ranging from about 15 to about 25° C. and a time period ranging from about 8 hours to about 24 hours.

In an illustrative embodiment, a GAG can be added to the reaction mixture in an amount ranging from about 20 wt. % to about 90 wt. %, based on the total weight of the reaction mixture. In one illustrative embodiment, a GAG can be added to the reaction mixture in an amount ranging from about 50 wt. % to about 80 wt. %, based on the total weight of the reaction mixture.

In an illustrative embodiment, a lifitegrast or a salt thereof can be added to the reaction mixture in an amount ranging from about 5 wt. % to about 50 wt. %, based on the total weight of the reaction mixture. In one illustrative embodiment, a lifitegrast or a salt thereof can be added to the reaction mixture in an amount ranging from about 10 wt. % to about 30 wt. 00 based on the total weight of the reaction mixture.

The lifitegrast or salt thereof-containing GAG polymer can be a random copolymer or a block copolymer. In one illustrative embodiment, a lifitegrast or a salt thereof-containing polymer disclosed herein can have a weight average molecular weight ranging from about 10,000 to about 6,000,000 Da in which the lower limit is from about 10,000, about 30,000, about 40,000, about 50,000, about 60,000, about 70,000, about 80,000, about 90,000, or about 100,000 Da, and the upper limit is about 100,000, about 150,000, about 200,000, about 300,000, about 400,000, about 500,000, about 600,000, about 700,000, about 800,000, about 900,000, about 1,000,000, about 2,000,000, about 3,000,000, about 4,000,000, about 5,000,000 or up to about 6,000,000 Da, wherein any of the lower limits can be combined with any of the upper limits.

A representative example of a reaction scheme to form a lifitegrast or a salt thereof-containing hyaluronic acid polymer can be seen below in Scheme II.

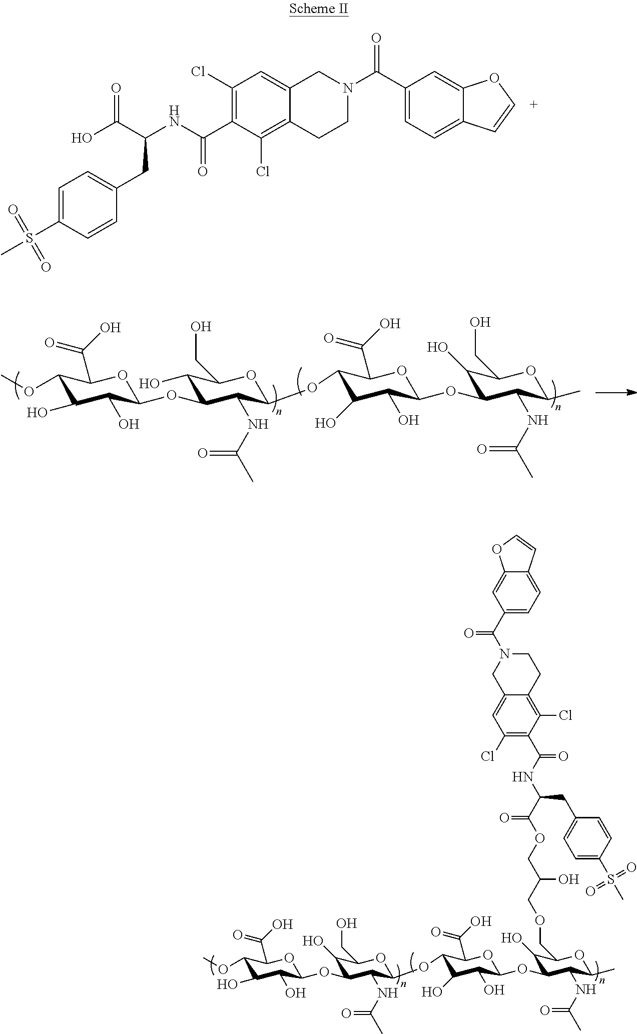

A representative example of a reaction scheme to form a lifitegrast or a salt thereof-containing hyaluronic acid polymer can be seen below in Scheme III.

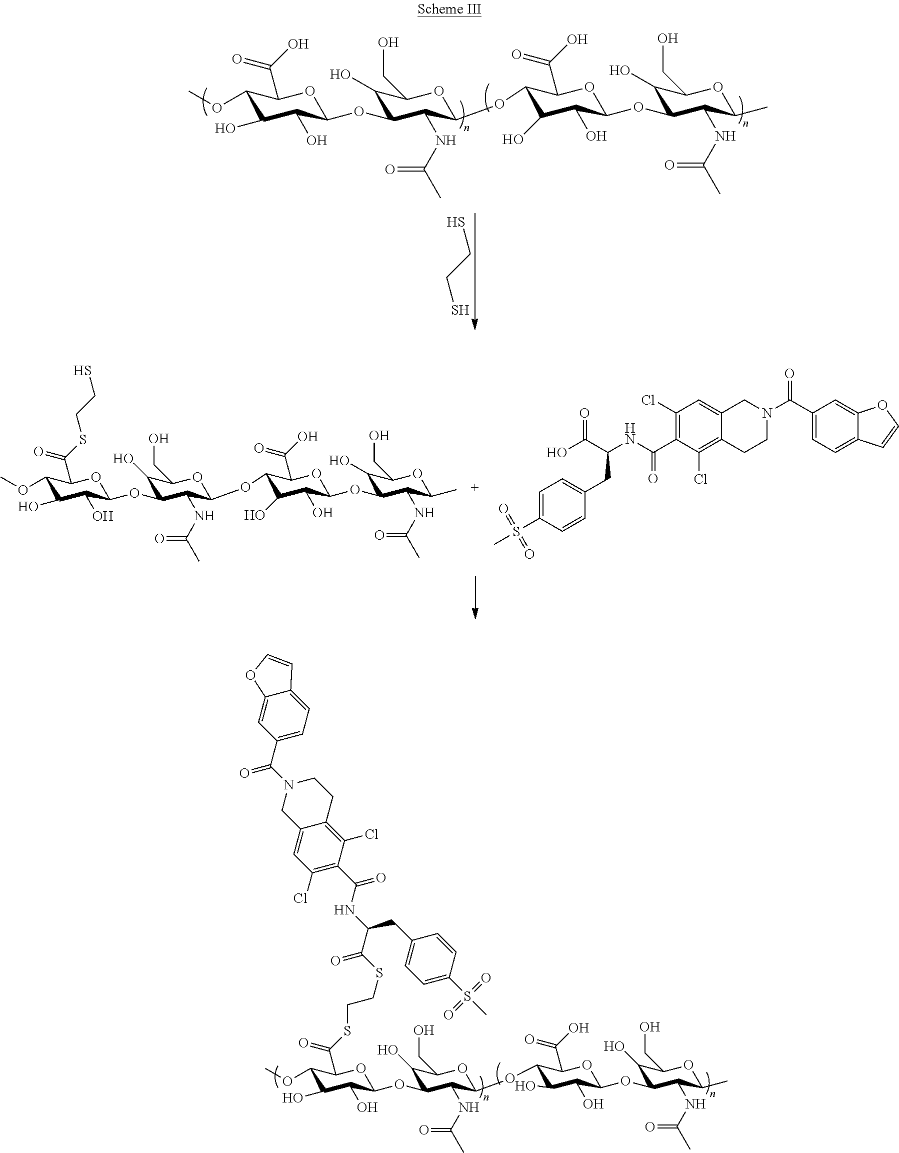

A representative example of a reaction scheme to form a lifitegrast or a salt thereof-containing hyaluronic acid polymer can be seen below in Scheme IV.

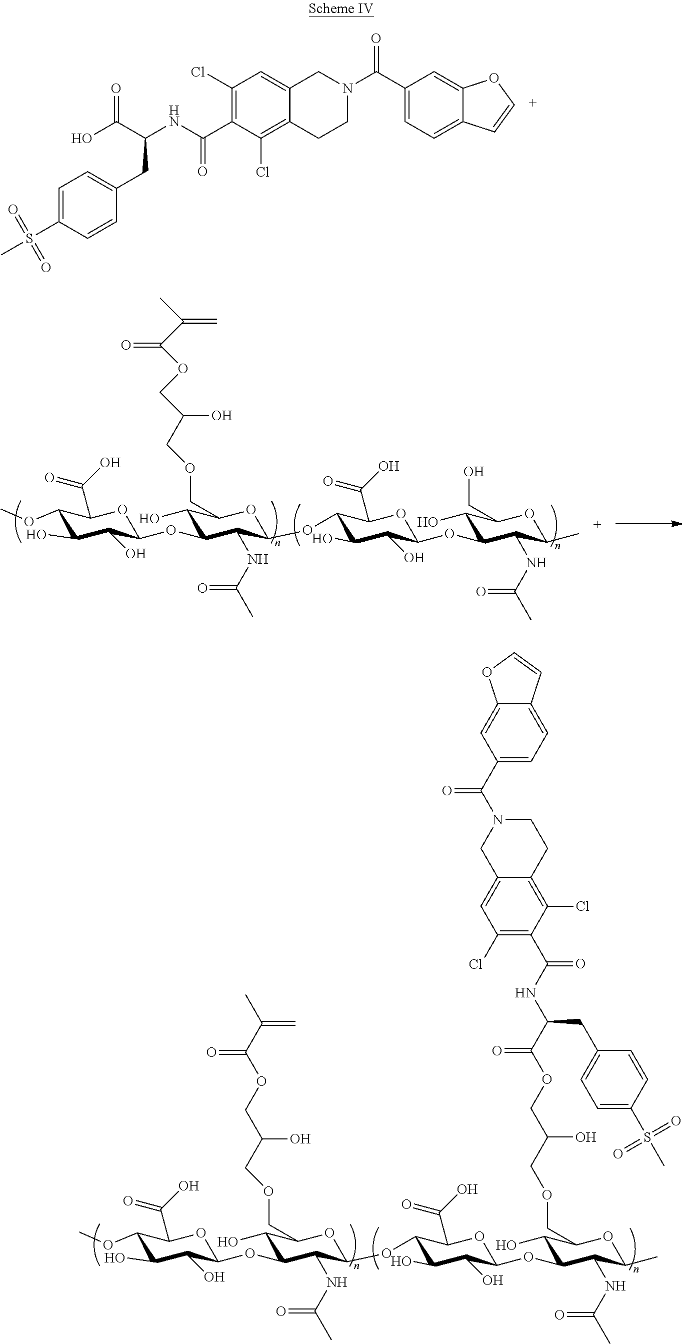

In some embodiments, the lifitegrast or salt thereof-containing polymer can be attached to the plurality of surface functional groups via one or more reactive functional groups in the polymer backbone of the lifitegrast or salt thereof-containing polymer. For example, one or more reactive functional groups in the polymer backbone of the lifitegrast or salt thereof-containing polymer can include, for example, a carboxylic acid group, or a hydroxyl group. Upon attaching the reactive functionality of the lifitegrast or salt thereof-containing polymer to the plurality of surface functional groups of the ophthalmic device, a releasable linking group can be formed that includes at least one bond that can be broken under physiological conditions, e.g., where the releasable linking group is a group linking the lifitegrast or salt thereof-containing polymer to the plurality of surface functional groups of the ophthalmic device via a pH-labile bond, an acid-labile bond, a base-labile bond, an oxidatively labile bond, a metabolically labile bond, a biochemically labile bond or an enzyme-labile bond, or other conditions such as a standard chemical reaction via a chemical-labile bond, e.g., a hydrolysis or substitution reaction, or via a photo-labile bond, e.g., exposure to light such as UV, near UV or visible light, e.g., a photosensitive bond. If the releasable linking groups are naturally cleaved under physiological conditions or cellular physiological conditions, then the polymer is biodegradable. In some embodiments, the releasable linking groups results in a loss of the lifitegrast or salt thereof-containing polymer from the surface of the ophthalmic device thereby releasing the lifitegrast or salt thereof-containing polymer.

In some embodiments, the releasable linking group includes ester groups. In the case of ester bonds, the esters undergo hydrolysis and are also catalytically cleaved by esterases thereby releasing the lifitegrast or salt thereof-containing polymer.

In some embodiments, the liftograft or salt thereof can be attached to the polymer via a photocleavable linking group. Suitable photocleavable linking groups include, for example, a nitrobenzyl group, phenacyl group, pyronin group, benzoin group and coumarin group. In one aspect, the photocleavable linking group can have at least two reactive groups. The first reactive group allows linking of the photocleavable linking group to an ethylenically unsaturated reactive group. The second reactive group allows linking of the photocleavable linking group to the lifitegrast or salt thereof. The former handle is preferably stable, and the latter handle is preferably amenable to photolysis such that the lifitegrast or salt thereof is released from the photocleavable lifitegrast conjugate upon exposure to light of the appropriate wavelength. The photocleavable group therefore breaks its bond with the lifitegrast or salt thereof, removing the charged groups from the lifitegrast or salt thereof. The lifitegrast or salt thereof is then in its native form with no additions.

A representative example of a reaction scheme to form a lifitegrast or a salt thereof-containing hyaluronic acid polymer with a releasable linking group that is a photocleavable can be seen below in steps 1 and 2 of Scheme V.

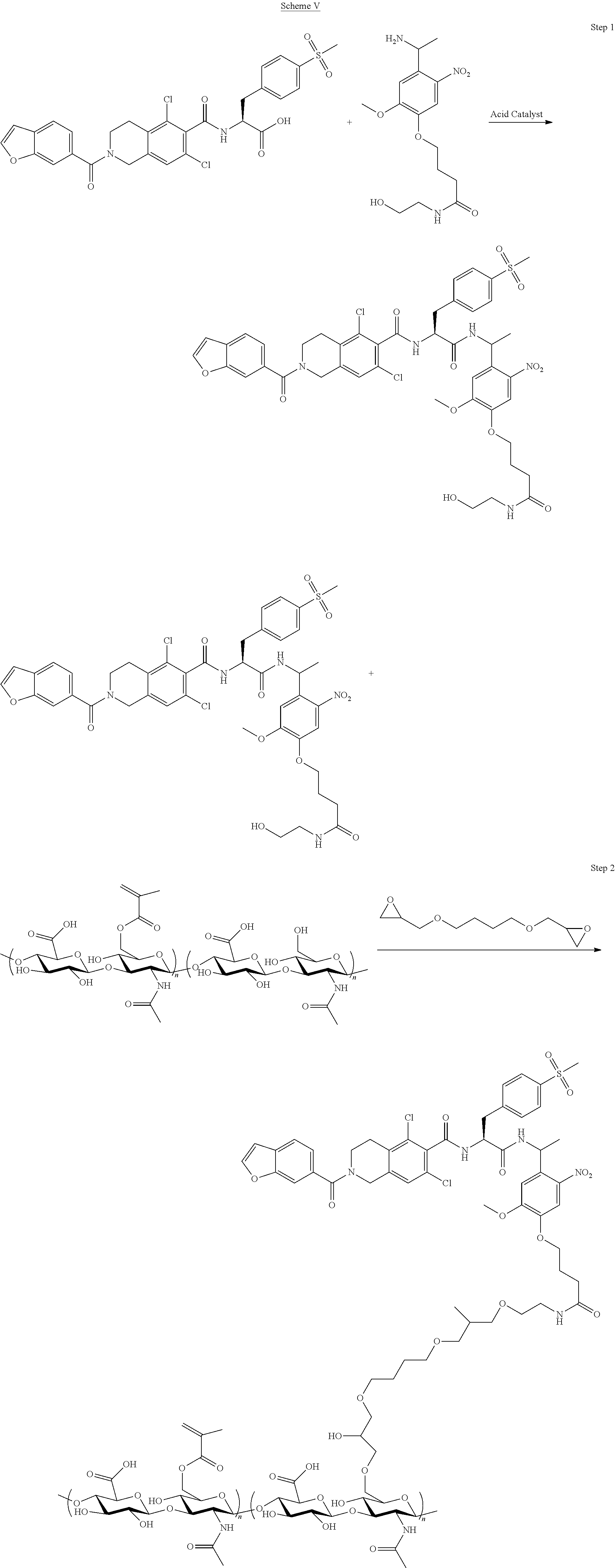

The lifitegrast or salt thereof-containing monomer or lifitegrast or salt thereof-containing polymer can be attached to the plurality of surface functional groups of the ophthalmic device by techniques known in the art. Suitable techniques include, for example, immersion, dip coating, spray coating, electrostatic coating and the like. For example, in one embodiment, the ophthalmic device can be contacted with a solution containing the lifitegrast or salt thereof-containing monomer or lifitegrast or salt thereof-containing polymer for a time period sufficient for the reactive groups of the lifitegrast or salt thereof-containing monomer or lifitegrast or salt thereof-containing polymer to covalently bond to the plurality of surface functional groups of the ophthalmic device at about room temperature or under autoclave conditions. In some embodiments, the coating solution can be an aqueous solution. Alternatively, the coating solution can contain an organic solvent, such as tetrahydrofuran, methanol, ethanol, isopropanol and the like and mixtures thereof.

The lifitegrast or salt thereof-containing monomer or lifitegrast or salt thereof-containing polymer can be retained on the surface of the ophthalmic device through an interaction of the lifitegrast or salt thereof-containing monomer or lifitegrast or salt thereof-containing polymer and the surface functional groups of the ophthalmic device. For example, the lifitegrast or salt thereof-containing monomer or lifitegrast or salt thereof-containing polymer may have chemical binding interactions between the plurality of surface functional groups of the ophthalmic device and the reactive groups of the lifitegrast or salt thereof-containing monomer or lifitegrast or salt thereof-containing polymer. Generally, the chemical binding interactions include, but are not limited to, ionic chemical interactions, covalent interactions, hydrogen-bond interactions, hydrophobic interactions, and hydrophilic interactions. Hydrogen-bonding interactions may involve hydrogen-bond donating groups or hydrogen bond accepting groups located on the surface of a biomedical device or as a chemical functional group moiety attached to the copolymer material. Alternatively, such an interaction can involve complexation between the coating copolymer and the biomedical-device surface functional groups. For example, the plurality of surface functional groups of the ophthalmic device such as an anhydride, lactone, carboxylic acid, acid chloride and/or epoxide, can react with, for example, carboxylic acid groups, carboxylate groups or alcohol groups of the lifitegrast or salt thereof-containing monomer or lifitegrast or salt thereof-containing polymer.

In some embodiments, a lifitegrast or a salt thereof-containing monomer or lifitegrast or salt thereof-containing polymer can be retained on the surface of the ophthalmic device by immersing an unused ophthalmic device having a plurality of surface functional groups in an aqueous packaging solution comprising a lifitegrast or a salt thereof-containing monomer or lifitegrast or salt thereof-containing polymer, packaging the aqueous packaging solution and the unused ophthalmic device in a manner preventing contamination of the unused ophthalmic device by microorganisms, and sterilizing the packaged solution and the unused ophthalmic device.

In some embodiments, a lifitegrast or a salt thereof-containing monomer or lifitegrast or salt thereof-containing polymer can be present in the aqueous packaging solution in an amount ranging from about 0.1 wt. % to about 5 wt. %, based on the total weight of the aqueous packaging solution. In some embodiments, a lifitegrast or a salt thereof-containing monomer or lifitegrast or salt thereof-containing polymer can be present in the aqueous packaging solution in an amount ranging from about 0.1 wt. % to about 2 wt. %, based on the total weigh of the aqueous packaging solution.

Appropriate packaging designs and materials are known in the art. A plastic package is releasably sealed with a film. Suitable sealing films are known in the art and include foils, polymer films and mixtures thereof. The sealed packages containing the lenses are then sterilized to ensure a sterile product. Suitable sterilization means and conditions are known in the art and include, for example, steam sterilizing or autoclaving of the sealed container at temperatures of about 120° C. or higher.

The aqueous packaging solutions of the illustrative embodiments are physiologically compatible. Specifically, the aqueous packaging solution must be “ophthalmically safe” for use with a lens such as a contact lens, meaning that a contact lens treated with the solution is generally suitable and safe for direct placement on the eye without rinsing, that is, the aqueous packaging solution is safe and comfortable for daily contact with the eye via a contact lens that has been wetted with the solution. An ophthalmically safe solution has a tonicity and pH that is compatible with the eye and includes materials, and amounts thereof, that are non-cytotoxic according to ISO standards and U.S. Food & Drug Administration (FDA) regulations.

The aqueous packaging solution should also be sterile in that the absence of microbial contaminants in the product prior to release must be statistically demonstrated to the degree necessary for such products. The liquid media useful in the present invention are selected to have no substantial detrimental effect on the lens being treated or cared for and to allow or even facilitate the present lens treatment or treatments. The liquid media are preferably aqueous-based. A particularly useful aqueous liquid medium is that derived from saline, for example, a conventional saline solution or a conventional buffered saline solution.

The pH of the aqueous packaging solutions is maintained within the range of about 6 to about 9, and preferably about 6.5 to about 7.8. As mentioned above, additional buffer may optionally be added, such as boric acid, sodium borate, potassium citrate, sodium citrate, citric acid, sodium bicarbonate, various mixed phosphate buffers (including combinations of Na₂ HPO₄, NaH₂ PO₄ and KH₂ PO₄), hydrates thereof and the like and mixtures thereof. Generally, buffers will be used in amounts ranging from about 0.05 to about 2.5 percent by weight, and preferably from about 0.1 to about 1.5 percent by weight of the solution. However, according to certain embodiments, tris(hydroxymethyl)aminomethane, or salts thereof, function as the sole buffer.

In one embodiment, the aqueous packaging solution can further comprise one or more buffer agents. Suitable one or more buffer agents include, for example, phosphate buffer agents, borate buffer agents, citrate buffer agents, and the like. A suitable phosphate buffer agent can be any known phosphate buffer agents. In one embodiment, the phosphate buffer agent comprises one or more of sodium hydrogen phosphate monobasic, sodium hydrogen phosphate dibasic, potassium hydrogen phosphate monobasic and potassium hydrogen phosphate dibasic and any suitable hydrate thereof, e.g., monohydrate and heptahydrate. A suitable borate buffer agent can be any known borate buffer agents. In one embodiment, the borate buffer agent comprises one or more of boric acid and sodium borate. A suitable citrate buffer agent can be any known citrate buffer agents. In one embodiment, the citrate buffer agent comprises one or more of citric acid and sodium citrate.

In one embodiment, the one or more buffer agents are present in the aqueous packaging solution in an amount ranging from about 0.001 wt. % to about 2 wt. %, based on the total weight of the packaging solution. In one embodiment, the phosphate buffer agent is present in the packaging solution in an amount ranging from about 0.001 wt. % to about 1 wt. %, based on the total weight of the packaging solution.

Typically, the aqueous packaging solutions are also adjusted with tonicity agents, to approximate the osmotic pressure of normal lacrimal fluids which is equivalent to a 0.9 percent solution of sodium chloride or 2.5 percent of glycerol solution. The solutions are made substantially isotonic with physiological saline used alone or in combination, otherwise if simply blended with sterile water and made hypotonic or made hypertonic the lenses will lose their desirable optical parameters. Correspondingly, excess saline may result in the formation of a hypertonic solution which will cause stinging and eye irritation.

Examples of suitable tonicity adjusting agents include, but are not limited to, sodium and potassium chloride, dextrose, glycerin, calcium and magnesium chloride and the like and mixtures thereof. These agents are typically used individually in amounts ranging from about 0.01 to about 2.5% w/v and preferably from about 0.2% w/v to about 1.5% w/v. Preferably, the tonicity agent will be employed in an amount to provide a final osmotic value of at least about 200 mOsm/kg, or from about 200 to about 400 mOsm/kg, or from about 250 to about 350 mOsm/kg, or from about 280 to about 320 mOsm/kg.

If desired, one or more additional components can be included in the packaging solution. Such an additional component or components are chosen to impart or provide at least one beneficial or desired property to the packaging solution. Such additional components may be selected from components which are conventionally used in one or more ophthalmic device care compositions. Examples of such additional components include cleaning agents, wetting agents, nutrient agents, sequestering agents, viscosity builders, contact lens conditioning agents, antioxidants, and the like and mixtures thereof. These additional components may each be included in the packaging solutions in an amount effective to impart or provide the beneficial or desired property to the packaging solutions. For example, such additional components may be included in the packaging solutions in amounts similar to the amounts of such components used in other, e.g., conventional, contact lens care products.

Useful sequestering agents include, but are not limited to, disodium ethylene diamine tetraacetate, alkali metal hexametaphosphate, citric acid, sodium citrate and the like and mixtures thereof.

Useful viscosity builders include, but are not limited to, hydroxyethyl cellulose, hydroxymethyl cellulose, polyvinyl pyrrolidone, polyvinyl alcohol and the like and mixtures thereof.

Useful antioxidants include, but are not limited to, sodium metabisulfite, sodium thiosulfate, N-acetylcysteine, butylated hydroxyanisole, butylated hydroxytoluene and the like and mixtures thereof.

In an illustrative embodiment, the steps leading to the packaging system disclosed herein include (1) molding an ophthalmic device in a mold comprising at least a first and second mold portion, (2) hydrating and cleaning the ophthalmic device in a container optionally comprising at least one of the mold portions, (3) introducing the aqueous packaging solution disclosed herein into the container with the ophthalmic device supported therein, (4) sealing the container, and (5) sterilizing the contents of the container. Sterilization may take place prior to, or most conveniently after, sealing of the container and may be affected by any suitable method known in the art, e.g., by autoclaving of the sealed container at temperatures of about 120° C. or higher.

According to an aspect of the present disclosure, a surface modified ophthalmic device comprises an ophthalmic device having a plurality of surface functional groups and a surface coating, the surface coating comprising a lifitegrast or a salt thereof-containing monomer or a lifitegrast or a salt thereof-containing polymer attached to a surface of the ophthalmic device through the plurality of surface functional groups.

In non-limiting illustrative embodiments, as may be combined with one or more of the preceding paragraphs, the plurality of surface functional groups are selected from the group consisting of a hydroxy group, amino group, carboxy group, carbonyl group, aldehyde group, sulfonic acid group, sulfonyl chloride group, isocyanato group, carboxy anhydride group, lactone group, azlactone group, epoxy group and mixtures thereof.

In non-limiting illustrative embodiments, as may be combined with one or more of the preceding paragraphs, the surface coating comprises the lifitegrast or salt thereof-containing monomer.

In non-limiting illustrative embodiments, as may be combined with one or more of the preceding paragraphs, the lifitegrast or salt thereof-containing monomer is attached to the plurality of surface functional groups via a pH-labile bond, an acid-labile bond, a base-labile bond, an oxidatively labile bond, a metabolically labile bond, a biochemically labile bond, an enzyme-labile bond, a chemical-labile bond or a photo-labile bond.

In non-limiting illustrative embodiments, as may be combined with one or more of the preceding paragraphs, the plurality of surface functional groups comprises one or more of a carboxylic acid group and a hydroxyl group and the lifitegrast or salt thereof-containing monomer is attached to the plurality of surface functional groups via a carboxylic acid group or a carboxylate group.

In non-limiting illustrative embodiments, as may be combined with one or more of the preceding paragraphs, the surface coating comprises the lifitegrast or salt thereof-containing polymer.

In non-limiting illustrative embodiments, as may be combined with one or more of the preceding paragraphs, the lifitegrast or salt thereof-containing polymer comprises a lifitegrast or a salt thereof-containing glycosaminoglycan polymer.

In non-limiting illustrative embodiments, as may be combined with one or more of the preceding paragraphs, the lifitegrast or salt thereof-containing polymer comprises lifitegrast or a salt thereof attached to the polymer via a releasable linking group.

In non-limiting illustrative embodiments, as may be combined with one or more of the preceding paragraphs, the releasable linking group attaching the lifitegrast or salt thereof to the polymer is one of a pH-labile bond, an acid-labile bond, a base-labile bond, an oxidatively labile bond, a metabolically labile bond, a biochemically labile bond, an enzyme-labile bond, a chemical-labile bond or a photo-labile bond.

In non-limiting illustrative embodiments, as may be combined with one or more of the preceding paragraphs, the lifitegrast or salt thereof-containing polymer comprises a glycosaminoglycan having a polymer backbone and a side chain comprising a reactive functionality complementary to a reactive functionality of lifitegrast or a salt thereof to provide a lifitegrast or a salt thereof residue grafted onto the polymer backbone.

In non-limiting illustrative embodiments, as may be combined with one or more of the preceding paragraphs, the glycosaminoglycan is hyaluronic acid or a salt thereof.

In non-limiting illustrative embodiments, as may be combined with one or more of the preceding paragraphs, the lifitegrast or salt thereof residue is grafted onto the polymer backbone via an ester bond.

In non-limiting illustrative embodiments, as may be combined with one or more of the preceding paragraphs, the lifitegrast or salt thereof residue is grafted onto the polymer backbone via a nitrobenzyl group.

In non-limiting illustrative embodiments, as may be combined with one or more of the preceding paragraphs, the ophthalmic device is an ophthalmic lens.

In non-limiting illustrative embodiments, as may be combined with one or more of the preceding paragraphs, the ophthalmic lens is a contact lens or an intraocular lens.

In non-limiting illustrative embodiments, as may be combined with one or more of the preceding paragraphs, the ophthalmic device is a silicone hydrogel continuous-wear lens.

According to another aspect of the present disclosure, a method for making a surface modified ophthalmic device comprises:
providing an ophthalmic device having a plurality of surface functional groups, and
attaching a lifitegrast or a salt thereof-containing monomer or a lifitegrast or a salt thereof-containing polymer to a surface of the ophthalmic device through the plurality of surface functional groups.

In non-limiting illustrative embodiments, as may be combined with one or more of the preceding paragraphs, the plurality of surface functional groups are selected from the group consisting of a hydroxy group, amino group, carboxy group, carbonyl group, aldehyde group, sulfonic acid group, sulfonyl chloride group, isocyanato group, carboxy anhydride group, lactone group, azlactone group, epoxy group and mixtures thereof.

In non-limiting illustrative embodiments, as may be combined with one or more of the preceding paragraphs, the lifitegrast or salt thereof-containing monomer is attached to the plurality of surface functional groups via a pH-labile bond, an acid-labile bond, a base-labile bond, an oxidatively labile bond, a metabolically labile bond, a biochemically labile bond, an enzyme-labile bond, a chemical-labile bond or a photo-labile bond.

In non-limiting illustrative embodiments, as may be combined with one or more of the preceding paragraphs, the plurality of surface functional groups comprises one or more of a carboxylic acid group and a hydroxyl group and the lifitegrast or salt thereof-containing monomer is attached to the plurality of surface functional groups via a carboxylic acid group or a carboxylate group.

In non-limiting illustrative embodiments, as may be combined with one or more of the preceding paragraphs, the lifitegrast or salt thereof-containing polymer comprises a lifitegrast or a salt thereof-containing glycosaminoglycan polymer.

In non-limiting illustrative embodiments, as may be combined with one or more of the preceding paragraphs, the lifitegrast or salt thereof-containing polymer comprises lifitegrast or a salt thereof attached to the polymer via a releasable linking group.

In non-limiting illustrative embodiments, as may be combined with one or more of the preceding paragraphs, the releasable linking group attaching the lifitegrast or salt thereof to the polymer is one of a pH-labile bond, an acid-labile bond, a base-labile bond, an oxidatively labile bond, a metabolically labile bond, a biochemically labile bond, an enzyme-labile bond, a chemical-labile bond or a photo-labile bond.

In non-limiting illustrative embodiments, as may be combined with one or more of the preceding paragraphs, the lifitegrast or salt thereof-containing polymer comprises a glycosaminoglycan having a polymer backbone and a side chain comprising a reactive functionality complementary to a reactive functionality of lifitegrast or a salt thereof to provide a lifitegrast or a salt thereof residue grafted onto the polymer backbone.

In non-limiting illustrative embodiments, as may be combined with one or more of the preceding paragraphs, the glycosaminoglycan is hyaluronic acid or a salt thereof.

In non-limiting illustrative embodiments, as may be combined with one or more of the preceding paragraphs, the lifitegrast or salt thereof residue is grafted onto the polymer backbone via an ester bond.

In non-limiting illustrative embodiments, as may be combined with one or more of the preceding paragraphs, the lifitegrast or salt thereof residue is grafted onto the polymer backbone via a nitrobenzyl group.

In non-limiting illustrative embodiments, as may be combined with one or more of the preceding paragraphs, the ophthalmic device is an ophthalmic lens.

In non-limiting illustrative embodiments, as may be combined with one or more of the preceding paragraphs, the ophthalmic lens is a contact lens or an intraocular lens.

In non-limiting illustrative embodiments, as may be combined with one or more of the preceding paragraphs, the ophthalmic device is a silicone hydrogel continuous-wear lens.

According to another aspect of the present disclosure, a method for delivering lifitegrast or salt thereof to an eye of a subject, comprises:
placing a surface modified ophthalmic device according to any one of the preceding paragraphs in an eye of a subject; and
delivering the lifitegrast or salt thereof to the eye of the subject by contacting the ophthalmic device with one of light having a wavelength of about 300 nanometers (nm) to about 700 nm or with an esterases or enzymes on the eye, in a tear film or on a flora of the eye.

According to another aspect of the present disclosure, a packaging system for storage of a surface modified ophthalmic device comprises a sealed container containing an unused ophthalmic device having a plurality of surface functional groups immersed in an aqueous packaging solution comprising a lifitegrast or a salt thereof-containing monomer or a lifitegrast or a salt thereof-containing polymer, wherein the aqueous packaging solution has an osmolality of at least about 150 mOsm/kg, a pH of about 6 to about 9 and is sterilized.

In non-limiting illustrative embodiments, as may be combined with one or more of the preceding paragraphs, the plurality of surface functional groups are selected from the group consisting of a hydroxy group, amino group, carboxy group, carbonyl group, aldehyde group, sulfonic acid group, sulfonyl chloride group, isocyanato group, carboxy anhydride group, lactone group, azlactone group, epoxy group and mixtures thereof.

In non-limiting illustrative embodiments, as may be combined with one or more of the preceding paragraphs, the aqueous packaging solution comprises the lifitegrast or salt thereof-containing monomer.

In non-limiting illustrative embodiments, as may be combined with one or more of the preceding paragraphs, the lifitegrast or salt thereof-containing monomer is attached to the plurality of surface functional groups via a pH-labile bond, an acid-labile bond, a base-labile bond, an oxidatively labile bond, a metabolically labile bond, a biochemically labile bond, an enzyme-labile bond, a chemical-labile bond or a photo-labile bond.

In non-limiting illustrative embodiments, as may be combined with one or more of the preceding paragraphs, the plurality of surface functional groups comprises one or more of a carboxylic acid group and a hydroxyl group and the lifitegrast or salt thereof-containing monomer is attached to the plurality of surface functional groups via a carboxylic acid group or a carboxylate group.

In non-limiting illustrative embodiments, as may be combined with one or more of the preceding paragraphs, the aqueous packaging solution comprises the lifitegrast or salt thereof-containing polymer.

In non-limiting illustrative embodiments, as may be combined with one or more of the preceding paragraphs, the lifitegrast or salt thereof-containing polymer comprises a lifitegrast or a salt thereof-containing glycosaminoglycan polymer.

In non-limiting illustrative embodiments, as may be combined with one or more of the preceding paragraphs, the lifitegrast or salt thereof-containing polymer comprises lifitegrast or a salt thereof attached to the polymer via a releasable linking group.

In non-limiting illustrative embodiments, as may be combined with one or more of the preceding paragraphs, the releasable linking group attaching the lifitegrast or salt thereof to the polymer is one of a pH-labile bond, an acid-labile bond, a base-labile bond, an oxidatively labile bond, a metabolically labile bond, a biochemically labile bond, an enzyme-labile bond, a chemical-labile bond or a photo-labile bond.

In non-limiting illustrative embodiments, as may be combined with one or more of the preceding paragraphs, the lifitegrast or salt thereof-containing polymer comprises a glycosaminoglycan having a polymer backbone and a side chain comprising a reactive functionality complementary to a reactive functionality of lifitegrast or a salt thereof to provide a lifitegrast or a salt thereof residue grafted onto the polymer backbone.

In non-limiting illustrative embodiments, as may be combined with one or more of the preceding paragraphs, the glycosaminoglycan is hyaluronic acid or a salt thereof.

In non-limiting illustrative embodiments, as may be combined with one or more of the preceding paragraphs, the lifitegrast or salt thereof residue is grafted onto the polymer backbone via an ester bond.

In non-limiting illustrative embodiments, as may be combined with one or more of the preceding paragraphs, the lifitegrast or salt thereof residue is grafted onto the polymer backbone via a nitrobenzyl group.

In non-limiting illustrative embodiments, as may be combined with one or more of the preceding paragraphs, the unused ophthalmic device is an unused ophthalmic lens.

In non-limiting illustrative embodiments, as may be combined with one or more of the preceding paragraphs, the unused ophthalmic lens is an unused contact lens or an unused intraocular lens.

In non-limiting illustrative embodiments, as may be combined with one or more of the preceding paragraphs, the unused ophthalmic device is an unused silicone hydrogel continuous-wear lens.

Various features disclosed herein are, for brevity, described in the context of a single embodiment, but may also be provided separately or in any suitable sub-combination. All combinations of the embodiments are specifically embraced by the illustrative embodiments disclosed herein just as if each and every combination was individually and explicitly disclosed. In addition, all sub-combinations listed in the embodiments describing such variables are also specifically embraced by the present compositions and are disclosed herein just as if each and every such sub-combination was individually and explicitly disclosed herein.

It will be understood that various modifications may be made to the embodiments disclosed herein. Therefore, the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the features and advantages appended hereto.

What is claimed is:

1. A surface modified ophthalmic device comprising an ophthalmic device having a plurality of surface functional groups and a surface coating, the surface coating comprising a lifitegrast or a salt thereof-containing monomer or a lifitegrast or a salt thereof-containing polymer attached to a surface of the ophthalmic device through the plurality of surface functional groups.

2. The surface modified ophthalmic device according to claim 1, wherein the plurality of surface functional groups are selected from the group consisting of a hydroxy group, amino group, carboxy group, carbonyl group, aldehyde group, sulfonic acid group, sulfonyl chloride group, isocyanato group, carboxy anhydride group, lactone group, azlactone group, epoxy group and mixtures thereof.

3. The surface modified ophthalmic device according to claim 1, wherein the lifitegrast or salt thereof-containing monomer is attached to the plurality of surface functional groups via a pH-labile bond, an acid-labile bond, a base-labile bond, an oxidatively labile bond, a metabolically labile bond, a biochemically labile bond, an enzyme-labile bond, a chemical-labile bond or a photo-labile bond.

4. The surface modified ophthalmic device according to claim 1, wherein the lifitegrast or salt thereof-containing polymer comprises a lifitegrast or a salt thereof-containing glycosaminoglycan polymer.

5. The surface modified ophthalmic device according to claim 4 wherein the lifitegrast or salt thereof-containing polymer comprises lifitegrast or a salt thereof attached to the polymer via a releasable linking group.

6. The surface modified ophthalmic device according to claim 5, wherein the releasable linking group attaching the lifitegrast or salt thereof to the polymer is one of a pH-labile bond, an acid-labile bond, a base-labile bond, an oxidatively labile bond, a metabolically labile bond, a biochemically labile bond, an enzyme-labile bond, a chemical-labile bond or a photo-labile bond.

7. The surface modified ophthalmic device according to claim 1, wherein the lifitegrast or salt thereof-containing polymer comprises a glycosaminoglycan having a polymer backbone and a side chain comprising a reactive functionality complementary to a reactive functionality of lifitegrast or a salt thereof to provide a lifitegrast or a salt thereof residue grafted onto the polymer backbone.

8. The surface modified ophthalmic device according to claim 7, wherein the glycosaminoglycan is hyaluronic acid or a salt thereof.

9. The surface modified ophthalmic device according to claim 7, wherein the lifitegrast or salt thereof residue is grafted onto the polymer backbone via an ester bond.

10. The surface modified ophthalmic device according to claim 7, wherein the lifitegrast or salt thereof residue is grafted onto the polymer backbone via a nitrobenzyl group.

11. The surface modified ophthalmic device according to claim 1, wherein the ophthalmic device is a contact lens or an intraocular lens.

12. A method for making a surface modified ophthalmic device comprising:
providing an ophthalmic device having a plurality of surface functional groups; and
attaching a lifitegrast or a salt thereof-containing monomer or a lifitegrast or a salt thereof-containing polymer to a surface of the ophthalmic device through the plurality of surface functional groups.

13. The method according to claim 12, herein the plurality of surface functional groups are selected from the group consisting of a hydroxy group, amino group, carboxy group, carbonyl group, aldehyde group, sulfonic acid group, sulfonyl chloride group, isocyanato group, carboxy anhydride group, lactone group, azlactone group, epoxy group and mixtures thereof.

14. The method according to claim 12, wherein the lifitegrast or salt thereof-containing monomer is attached to the plurality of surface functional groups via a pH-labile bond, an acid-labile bond, a base-labile bond, an oxidatively labile bond, a metabolically labile bond, a biochemically labile bond, an enzyme-labile bond, a chemical-labile bond or a photo-labile bond.

15. The method according to claim 12, wherein the lifitegrast or salt thereof-containing polymer comprises a lifitegrast or a salt thereof-containing glycosaminoglycan polymer.

16. The method according to claim 15, wherein the lifitegrast or salt thereof-containing polymer comprises lifitegrast or a salt thereof attached to the polymer via a releasable linking group.

17. The method according to claim 12, wherein the lifitegrast or salt thereof-containing polymer comprises a glycosaminoglycan having a polymer backbone and a side chain comprising a reactive functionality complementary to a reactive functionality of lifitegrast or a salt thereof to provide a lifitegrast or a salt thereof residue grafted onto the polymer backbone.

18. The method according to claim 17, wherein the glycosaminoglycan is hyaluronic acid or a salt thereof and the lifitegrast or salt thereof residue is grafted onto the polymer backbone via an ester bond or via a nitrobenzyl group.

19. The method according to claim 12, wherein the ophthalmic device is a contact lens or an intraocular lens.

20. A method for delivering lifitegrast or salt thereof to an eye of a subject, comprising:
placing a surface modified ophthalmic device in an eye of a subject, wherein the surface modified ophthalmic device comprises an ophthalmic device having a plurality of surface functional groups and a surface coating, the surface coating comprising a lifitegrast or a salt thereof-containing monomer or a lifitegrast or a salt thereof-containing polymer attached to a surface of the ophthalmic device through the plurality of surface functional groups; and
delivering the lifitegrast or salt thereof to the eye of the subject by contacting the ophthalmic device with one of light having a wavelength of about 300 nanometers (nm) to about 700 nm or with an esterases or enzymes on the eye, in a tear film or on a flora of the eye.
